(19) United States
(12) Patent Application Publication
(10) Pub. No.: US 2011/0306680 A1
(43) Pub. Date: Dec. 15, 2011

(54) COMPOSITIONS CONTAINING AGAR AND ESTERIFIED SUGAR SURFACTANTS

(75) Inventors: Gisela Perruna, Rahway, NJ (US); Dhaval Patel, Edison, NJ (US); Hy Si Bui, Piscataway, NJ (US)

(73) Assignee: L'Oreal S.A., Paris (FR)

(21) Appl. No.: 13/154,793

(22) Filed: Jun. 7, 2011

Related U.S. Application Data
(60) Provisional application No. 61/352,882, filed on Jun. 9, 2010.

(51) Int. Cl. A61K 47/36; A61Q 17/04; A01N 25/02; A61Q 19/08; A01P 17/00; A61Q 15/00; C08L 5/12; A61P 17/10

(52) U.S. Cl. 514/777; 106/205.71

(57) ABSTRACT

The present invention is directed to a composition that includes (a) at least one thermo-reversible polysaccharide chosen from agar; (b) at least one softening agent chosen from an esterified sugar surfactant; (c) at least one oil; (d) water; and (e) optionally, at least one emulsifier different from (b), as well as to processes for softening an emulsion containing agar by adding to the emulsion at least one softening agent chosen from an esterified sugar surfactant.

### CROSS-REFERENCE TO RELATED APPLICATIONS

This application claims the benefit of priority under 35 U.S.C. §119(e) from U.S. Provisional Application Ser. No. 61/352,882, filed Jun. 9, 2010, the entire contents of which is hereby incorporated by reference.

### FIELD OF THE INVENTION

The current invention is directed toward compositions, preferably personal care compositions, containing a material which is organic, sustainable and thermo-reversible.

### BACKGROUND

Agar is a known thermo-reversible polysaccharide derived from red algae, also known as seaweed. It is utilized in the pharmaceutical industry and in the food industry as a gellant or thickener when used in association with water.

The use of agar as a gellant is known to produce very rigid/hard gelled compositions. Such rigidity prevents the product from being easily retrieved from its container by either an applicator or a consumer's finger, a phenomenon commonly referred to in the industry as poor “pickup”.

The use of agar is also known to oftentimes result in the formation of craters on a resultant product's surface which is undesirable from an aesthetic standpoint. Moreover, the breaking of the product's surface during retrieval from its container oftentimes causes the product to crumble.

Therefore, a need exists to be able to incorporate agar into personal care compositions without the attendant problems of extreme rigidity, poor pickup, crater formation and crumbling.

### SUMMARY OF THE INVENTION

The present invention is directed to a composition comprising: (a) at least one thermo-reversible polysaccharide chosen from agar; (b) at least one softening agent chosen from an esterified sugar surfactant; (c) at least one oil; (d) water; and (e) optionally, at least one emulsifier different from (b).

The present invention is also directed to a process for softening an emulsion containing agar by adding to the emulsion: a) at least one softening agent chosen from an esterified sugar surfactant; and b) optionally, at least one emulsifier different from (a).

The present invention is also directed to a process for making a composition comprising mixing together: (a) at least one thermo-reversible polysaccharide chosen from agar; (b) at least one softening agent chosen from an esterified sugar surfactant; (c) at least one oil; (d) water; and (e) optionally, at least one emulsifier different from (b). Preferably, the composition has a soft texture, good pickup and a desirable aesthetic appearance.

It has been surprisingly found that by employing a specific type of compound as a softening agent in an emulsion containing agar, the resultant composition possesses a soft texture, resistance to crumbling and improved pickup.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

Generally, the present invention relates to emulsions, preferably oil-in-water emulsions, that use agar as a gelling agent. It has been surprisingly found that the associations of agar with certain types of softening agents, described herein, provide unique advantages. Particularly preferred softening agents include esterified sugar surfactants.

Without intending to be bound by theory, it is believed that when agar is used to gellify water-containing compositions, numerous hydrogen bonds are formed between the agar and the water molecules, resulting in the compositions becoming undesirably rigid, thereby yielding poor pickup, surface cratering and product crumbling. Adding the subject softening agents addresses such problems associated with agar-containing compositions.

Agar

According to the present invention, compositions comprising agar are provided. Chemically, agar is a polymer made up of subunits of the sugar galactose. Agar consists of a mixture of agarose and agaropectin. Agarose is a linear polymer, having a molecular weight of about 120,000. Agaropectin is a heterogeneous mixture of smaller molecules that occur in lesser amounts. Their structures are similar but slightly branched and sulfated, and they may have methyl and pyruvic acid ketal substituents.

Suitable agars include, for example, pharmaceutical grade agars. Pharmaceutical grade agar is defined as having been tested for safety, potency, purity and efficacy.

A preferred agar is AGAR AGAR IRX 23900, commercially available from CNI-Colloides Naturels International. Other preferred agars are Agar RS-100, Agar Agar 100 FCC/NF Powder, Agar Agar 150 FCC/NF Powder, all commercially available from TIC Gums. Still another preferred agar is Agar Agar Thermically Treated, commercially available from Setal G. A preferred pharmaceutical grade agar is Pharamaceutical Agar, commercially available from TIC Gums.

Preferably, the agar is employed in an amount ranging from about 0.1 to about 3% by weight, preferably from about 0.15 to about 2% by weight, and preferably from about 0.2 to about 1.0% by weight, including all ranges and subranges therebetween, all weights based on the total weight of the composition.

Softening Agent

According to the present invention, compositions comprising at least one softening agent (to soften compositions containing agar) are provided. Softening agents of the present invention are typically chosen from esterified sugar surfactants. Suitable esterified sugar surfactants include, but are not limited to, sucrose stearate and dextrin palmitate, and mixtures thereof.

Particularly preferred esterified sugar surfactants include sucrose stearate commercially available under the tradename RYOTO® SUGAR ESTER S 1570 and dextrin palmitate commercially available under the tradename RHEOPEARL® TL2-OR.

Preferably, the softening agent is employed in an amount sufficient to soften a composition containing agar (a softening effective amount), preferably ranging from about 0.1 to about 5% by weight, preferably from about 0.2 to about 4% by weight, and preferably from about 0.5 to about 2% by weight, including all ranges and subranges therebetween, all weights based on the total weight of the composition.

Oils

The composition of the present invention also contains at least one oil. Any oil can be used in accordance with the present invention. The oil can be volatile or non-volatile, silicone-based and/or hydrocarbon-based, etc. Thus, for example, the oil component may contain, independently or in combination, volatile silicone oils, non-volatile silicone oils, volatile non-silicone oils and non-volatile non-silicone oils.

According to one embodiment, the oil phase may contain one or more volatile silicone oils. Examples of such volatile silicone oils include linear or cyclic silicone oils having a viscosity at room temperature less than or equal to 6 cSt and having from 2 to 7 silicone atoms, these silicones being optionally substituted with alkyl or alkoxy groups of 1 to 10 carbon atoms. Suitable oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash | |
|---|---|---|
| | Point | Viscosity |
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. The volatility of the solvents/oils can be determined using the evaporation speed as set forth in U.S. Pat. No. 6,338,839, also incorporated herein by reference.

Examples of other silicone oils that may be used in the invention include non-volatile linear polydimethylsiloxanes (PDMSs), that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxydiphenylsiloxanes, diphenyl dimethicones, diphenyl methyldiphenyl trisiloxanes and 2-phenylethyl trimethylsiloxysilicates.

According to other preferred embodiments, the oil phase may contain one or more non-silicone volatile oils and may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C8 to C16 alkanes such as C8 to C16 isoalkanes (also known as isoparaffins), isododecane, isodecane, isohexadecane, and for example, the oils sold under the trade names of Isopar or Permethyl, the C8 to C16 branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether | 46 |
| acetate | |
| Isopar L (isoparaffin C₁₁-C₁₃) | 62 |
| Isopar H (isoparaffin C₁₁-C₁₂) | 56 |

Examples of other non-silicone oils which can be used in the compositions of the present invention include polar oils such as:

hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names MIGLYOL 810, 812 and 818 by the company Dynamit Nobel;

synthetic oils or esters of formula R5COOR6 in which R5 represents a linear or branched higher fatty acid residue containing from 1 to 40 carbon atoms, including and better still from 7 to 19 carbon atoms, and R6 represents a branched hydrocarbon-based chain containing from 1 to 40 carbon atoms, including and better still from 3 to 20 carbon atoms, with R6+R7, such as, for example, Purcellin oil (cetostearyl octanoate), isononyl isononanoate, C12 to C15 alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;

synthetic ethers containing from 10 to 40 carbon atoms;

C8 to C26 fatty alcohols, for instance oleyl alcohol; and

mixtures thereof.

In an embodiment of the present invention, a preferred oil is a non-volatile ester, more preferably, octyl palmitate.

Preferably, the oil is employed in an amount ranging from about 1.0 to about 35% by weight, preferably from about 5 to about 25% by weight, and more preferably from about 10 to about 15% by weight, including all ranges and subranges therebetween, all weights based on the total weight of the composition.

Water

The composition of the present invention also contains water in order to form an emulsion, preferably an oil-in-water emulsion.

Thus, for example, in an oil-in-water emulsion, the water is typically present in an amount of from about 40 to about 80% by weight, preferably from about 45 to about 70% by weight, and more preferably from about 50 to about 60% by weight, including all ranges and subranges therebetween, all weights based on the total weight of the composition.

It should be noted, however, that while the composition of the present invention is preferably in the form of an oil-in-water emulsion, the invention is intended to encompass water-in-oil emulsions, multiple emulsions, nanaoemulsions, etc. as well. Thus, for example, in the case of water-in-oil emulsions, the water is present in an amount of from about 20 to about 50% by weight, preferably from about 22 to about 45% by weight, and more preferably from about 25% to about 40% by weight, including all ranges and subranges therebetween, all weights based on the total weight of the composition.

Optional Ingredients

Emulsifiers

If desired, the composition may include an emulsifier, different from the above-described softening agent, which can also act as an emulsifier. Examples of suitable emulsifiers include: fatty acids, fatty alcohols, polyethoxylated fatty alcohols or polyglycerolated fatty alcohols, such as polyethoxylated stearyl alcohols or cetylstearyl alcohols; C16-C30 fatty acids neutralized by amines, ammonia or the alkali metal salts thereof; quaternary amines, amine oxides and amines, e.g., alkyl amines, alkyl imidazolines, ethoxylated amines, quaternary compounds, and quaternized esters.

Phospholipid emulsifiers can also be used herein. A phospholipid is a compound which on hydrolysis yields phosphoric acid, an alcohol, fatty acid, and a nitrogenous base. They are widely distributed in nature and include such substances as lecithin, cephalin, and sphingomyelin. Further, alkyl modified silicone emulsifiers may also be used. Examples include, but are not limited to, PEG-9 polydimethylsiloxyethyl dimethicone, PEG/PPG-10/3 oleyl ether dimethicone and PEG-9 polydimethylsiloxyethyl dimethicone.

If present, the emulsifier is employed in an amount ranging from about 0.5 to about 5% by weight, preferably from about 1 to about 4% by weight, and more preferably from about 1.5 to about 3% by weight, including all ranges and subranges therebetween, all weights based on the total weight of the composition.

Auxiliaries

Various types of auxiliary ingredients may be used in the composition of the present invention. Suitable active ingredients include, for example, co-emulsifiers; film-forming polymers; co-solvents; colorants such as pigments, inks and lakes; dermatological ingredients such as sunscreen agents, anti-acne agents, anti-aging compounds; insect repelling agents; transdermal pharmaceutical compounds; deodorant and antiperspirant agents; perfumes; dye compounds; etc. The type and amount of optional ingredient to be employed will depend on the composition's ultimate use, and is to be determined by those of ordinary skill in the art.

Personal care compositions which may be formulated using the composition of the present invention can include, but are not limited to, colored cosmetics such as foundation, eyeshadow, blush; deodorants; antiperspirants; lotions; sunscreens; moisturizers; and the like.

According to preferred embodiments, methods of softening a composition comprising agar which comprise adding at least one softening agent chosen from an esterified sugar surfactant are also provided. Optionally, the methods further comprise at least one emulsifier different from the esterified sugar surfactant.

According to preferred embodiments, methods of making the invention compositions are also provided. These methods comprise mixing together: (a) at least one thermo-reversible polysaccharide chosen from agar; (b) at least one softening agent chosen from an esterified sugar surfactant; (c) at least one oil; (d) water; and (e) optionally, at least one emulsifier different from (b). Preferably, the composition is prepared such that it has a soft texture, good pickup and a desirable aesthetic appearance.

The present invention is further described in terms of the following non-limiting example. Unless otherwise indicated, all parts and percentages are on a weight-by-weight percentage basis.

### EXAMPLE

| | | % |
|---|---|---|
| Phase | CTFA Name | wt/wt |
| A | Di Water | 57.61 |
| | Agar | 1 |
| | Sucrose Stearate | 1 |
| | Disodium EDTA | 0.05 |
| | PEG-7 Glyceryl Cocoate | 1 |
| | UnTreated Pigments | 15 |
| | Mica | 2 |
| | Humectants | 5.57 |
| | Preservatives | 0.77 |
| B | Octyl Palmitate | 13 |
| | Cetyl Alcohol | 0.5 |
| | MONTANOV ®: C14-22 Alcohol and | 2 |
| | C12-20 Alkylglucoside | |
| C | Silica | 0.5 |
| | TOTAL | 100 |

### Procedure

1. Added phase A to the main kettle and heated to 90° C. while grinding.
2. In a separate beaker, heated phase B to 75-80° C. and mixed until uniform.
3. Combined phase A and phase B while homogenizing at 75-80° C.
4. Added phase C to main kettle and mixed.

### 5. Cooled to 40° C.

The above-identified composition had a rich, creamy texture/light feel, good pickup and desirable aesthetic properties. In addition, the composition's reduced rigidity allowed the cited optional ingredients to be easily blended therewith.

1) A composition, comprising:
a) at least one thermo-reversible polysaccharide chosen from agar;
b) at least one softening agent chosen from an esterified sugar surfactant;
c) at least one oil; and
d) water.

2) The composition of claim 1, wherein (a) is employed in an amount of from 0.1 to 3% by weight, based on the weight of the composition.

3) The composition of claim 1, wherein (b) is chosen from sucrose stearate, dextrin palmitate, and mixtures thereof.

4) The composition of claim 1, wherein (b) is sucrose stearate.

5) The composition of claim 1, wherein (b) is dextrin palmitate.

6) The composition of claim 1, wherein (b) is employed in an amount of from 0.1 to 5% by weight, based on the weight of the composition.

7) The composition of claim 1, wherein (c) is octyl palmitate.

8) The composition of claim 1, wherein (c) is employed in an amount of from 1 to 35% by weight, based on the weight of the composition.

9) The composition of claim 1, in the form of an emulsion.

10) A process for softening an emulsion containing agar, comprising adding to the emulsion at least one softening agent chosen from an esterified sugar surfactant.

11) The process of claim 10, wherein the agar is present in the emulsion an amount of from 0.1 to 3% by weight, based on the weight of the emulsion.

12) The process of claim 10, wherein the esterified sugar surfactant is chosen from sucrose stearate, dextrin palmitate, and mixtures thereof.

13) The process of claim 10, wherein the esterified sugar surfactant is sucrose stearate.

14) The process of claim 10, wherein the esterified sugar surfactant is dextrin palmitate.

15) The process of claim 10, wherein the esterified sugar surfactant is employed in the emulsion in an amount of from 0.1 to about 5% by weight, based on the weight of the emulsion.

16) A process for making a composition comprising mixing together:
a) at least one thermo-reversible polysaccharide chosen from agar;
b) at least one softening agent chosen from an esterified sugar surfactant;
c) at least one oil; and
d) water.

17) The process of claim 16, wherein the agar is added in an amount of from 0.1 to 3% by weight, based on the weight of the composition.

18) The process of claim 16, wherein the esterified sugar surfactant is chosen from sucrose stearate, dextrin palmitate, and mixtures thereof.

19) The process of claim 16, wherein the esterified sugar surfactant is sucrose stearate.

20) The process of claim 16, wherein the esterified sugar surfactant is dextrin palmitate.

21) The process of claim 16, wherein the esterified sugar surfactant is added in an amount of from 0.1 to 5% by weight, based on the weight of the composition.

22) The process of claim 16, wherein oil is added in an amount of from 1 to 35% by weight, based on the weight of the composition.
